(19) United States
(12) Patent Application Publication
(10) Pub. No.: US 2009/0130696 A1
(43) Pub. Date: May 21, 2009

(54) HDL Cholesterol Sensor Using Selective Surfactant

(76) Inventors: Lindy Murphy, Yarnton (GB); Carla Burrows, Yarnton (GB); Simon Bayly, Yarnton (GB); Katherine Wilkinson, Yarnton (GB); Herbert Frank Askew, Yarnton (GB); Howard Orman, Yarnton (GB)

Correspondence Address: QUARLES & BRADY LLP, 411 E. WISCONSIN AVENUE, SUITE 2040, MILWAUKEE, WI, 53202-4497, US

(21) Appl. No.: 12/298,881

(22) PCT Filed: May 14, 2007
(86) PCT No.: PCT/GB2007/001764

§ 371 Date: Oct. 28, 2008

(30) Foreign Application Priority Data
May 12, 2006 (GB) 0609494.0

(51) Int. Cl. C12Q 1/60; G01N 33/92; C12Q 1/26; G01N 27/26; C12Q 1/32

(52) U.S. Cl. 435/11; 436/71; 435/25; 435/26; 204/435; 205/787

(57) ABSTRACT

A method for the determination of the amount of cholesterol in high density lipoproteins in a high density lipoprotein containing sample, said method comprising reacting the sample with a surfactant which preferentially reacts with high density lipoproteins in the sample, said surfactant being selected from hydroxyethyl glucamide derivatives and N-acyl-N-methyl glucamine derivatives, and measuring the amount of cholesterol in the high density lipoproteins, for example using an electrochemical technique.

### FIELD OF THE INVENTION

The present invention relates to a method for determining the amount of cholesterol bound to high density lipoproteins (HDL cholesterol) in a high density lipoprotein-(HDL-) containing sample. The invention also relates to a composition and a kit for use in such a method.

### BACKGROUND TO THE INVENTION

Many epidemiological investigations have demonstrated the strong and independent inverse association of high density lipoprotein (HDL), measured in terms of either its cholesterol or apo A1 content, to risk of coronary artery disease (CAD). It is said that the risk of CAD increases 2-3% for every 10 mg/L decrease in HDL cholesterol. Thus, higher HDL cholesterol concentrations are considered protective. The measurement of HDL cholesterol in characterizing risk for CAD and managing treatment of dyslipidemia has therefore become increasingly common in clinical laboratories.

Initial laboratory methods for HDL cholesterol measurement, adapted from research techniques, required a manual separation step with precipitation reagents, followed by analysis of the cholesterol content, most often by an automated chemistry analyzer. Typical separation steps involved the reaction of a precipitation reagent with low density lipoproteins (LDL), very low density lipoproteins (VLDL) and chylomicrons (CMV) in order to form an aggregate of these components. The aggregate was then removed from the reaction vessel, for example by centrifugation, leaving an HDL-containing sample ready for analysis. Separation of the precipitate was essential in order that the precipitate did rot interfere with the UV/Vis or colorimetrc analysis techniques used.

More recently, a number of techniques have been developed which do not require prior separation of the various lipoprotein fractions. These methods have the advantage that a measurement can typically be achieved in a single step, or at least without the need for precipitation to be carried out. Automation of the measurement is therefore possible. In one such approach, certain surfactants are used which break down the various lipoprotein fractions at different rates. For example, a surfactant might initially react more quickly with LDL, and reaction with HDL might occur more slowly. By measurement of the cholesterol content at a specified time after addition of the surfactant, the measurement has been found to have a greater dependency on the HDL cholesterol content than on the LDL cholesterol content.

This approach, however, has not generated the required accuracy and reliability in its results and the measurements made still retain some degree of dependency on the content of cholesterol in LDL, VLDL, and CM. A new approach is therefore required which provides a simple and yet reliable and accurate method for the measurement of the HDL cholesterol content of body fluids such as blood and plasma. The measurement should also have a reduced dependency, or be entirely independent, of the content of cholesterol bound to LDL, VLDL and CM in the test sample. Further, preferred methods will not employ specialist equipment, or require trained technicians to carry out.

### SUMMARY OF THE INVENTION

The present invention provides a method for the determination of the amount of cholesterol in high density lipoproteins in a high density lipoprotein containing sample, said method comprising reacting the sample with (a) a surfactant which preferentially breaks down high density lipoproteins, said surfactant being selected from hydroxyethyl glucamide derivatives and N-acyl-N-methyl glucamine derivatives, and measuring the amount of cholesterol in the high density lipoproteins. The surfactants used in the present invention have a very high preference for HDL over LDL, VLDL and CM. Whilst previous surfactants have been shown to react at different rates with HDL compared with other lipoproteins, the surfactants of the present invention react almost exclusively with HDL, and do not react, or substantially do not react, with other lipoproteins. It is believed that HDL in the sample is solubilised leaving HDL cholesterol available for reaction, whilst cholesterol bound to other lipoprotein fractions remains bound within the lipoprotein structure and is unavailable for reaction. The present invention is not, however, bound by this mode of action. An alternative theory is that the surfactants of the invention are those which preferentially enable HDL cholesterol to react in a cholesterol assay, whilst LDL cholesterol is substantially unable to react. The subsequent measurement of the cholesterol content of the sample is thus reflective of the HDL-cholesterol content only, and is substantially independent of the amount of cholesterol contained within other lipoprotein fractions. The method of the present invention is therefore highly selective for HDL and provides an accurate and reliable test for HDL-cholesterol.

The method of the invention has the further advantage of improved simplicity compared with prior art tests. An HDL cholesterol measurement can be obtained by reacting a sample with a single reagent mixture and making a single measurement of the cholesterol content. Further, a result can be obtained in a very short period of time, typically within a minute or a few minutes of addition of the sample.

The measurement of HDL cholesterol is typically carried out by reacting the sample with a cholesterol ester hydrolysing reagent and either cholesterol oxidase or cholesterol dehydrogenase. The present invention accordingly also provides a reagent mixture for use in a method for the determination of the amount of cholesterol in high density lipoproteins in a high density lipoprotein containing sample, the reagent mixture comprising
(a) a surfactant as defined herein which preferentially breaks down high density lipoproteins;
(b) a cholesterol ester hydrolysing reagent; and
(c) cholesterol oxidase or cholesterol dehydrogenase.

Also provided is a kit for the determination of the amount of cholesterol in high density lipoproteins in a high density lipoprotein containing sample, the kit comprising (a) a surfactant as defined herein which preferentially breaks down high density lipoproteins, (b) a cholesterol ester hydrolysing reagent, and (c) cholesterol oxidase or cholesterol dehydrogenase, and means for measuring the amount of cholesterol which reacts with the cholesterol oxidase or cholesterol dehydrogenase. The kit is typically an electrochemical device wherein the means for measuring the amount of cholesterol which reacts with the cholesterol oxidase or cholesterol dehydrogenase comprises
an electrochemical cell having a working electrode, a reference or pseudo reference electrode and optionally a separate counter electrode;
a power supply for applying a potential across the cell; and
a measuring instrument for measuring the resulting electrochemical response.

The present invention also provides a method of operating the kit of the invention, said method comprising
(i) contacting (1) the reagents (a), (b) and (c) and (2) a high density lipoprotein containing sample, with each other and with the electrodes;
(ii) applying a potential across the electrochemical cell; and
(iii) electrochemically detecting the amount of product formed by measuring the resulting electrochemical response.

The surfactants used in the invention have the particular advantage of improving the reproducibility of the HDL cholesterol determination. It has been found that some differentiation between the HDL and other lipoproteins can be achieved using certain esterases or lipases alone, or with surfactants such as CHAPs or deoxyBIGCHAP. However, determinations on patient samples have been found to be highly variable. The use of surfactants described in this invention have been found to significantly reduce this variability. This is best described in relation to standard statistical measures of comparison, for example an improved R² value of the calibration line where HDL concentration is related to electrode current, although it can also be shown using other statistical techniques, particularly multiple regression analysis.

It is thus evident that the surfactants of the invention reduce the variability inherent in multiple tests. This reduced variability results in improved precision and accuracy in the assay. The reduced variability also leads the invention to find particular use where a number of different test results are to be compared, e.g. in the monitoring of a single patient's cholesterol levels over time.

The invention therefore also provides the use of the surfactants defined herein in improving the reproducibility (e.g. improving the R² value of the calibration line where HDL concentration is related to electrode current) of a method for the determination of the amount of cholesterol in high density lipoproteins. Also provided is a method of improving the reproducibility (e.g. improving the R² value of the calibration line where HDL concentration is related to electrode current) of a determination of the amount of HDL cholesterol in a sample, which comprises reacting the sample with (a) a surfactant as defined herein and measuring the amount of cholesterol in the high density lipoproteins.

### BRIEF DESCRIPTION OF THE FIGURES

FIG. 1 depicts a device according to an embodiment of the invention.

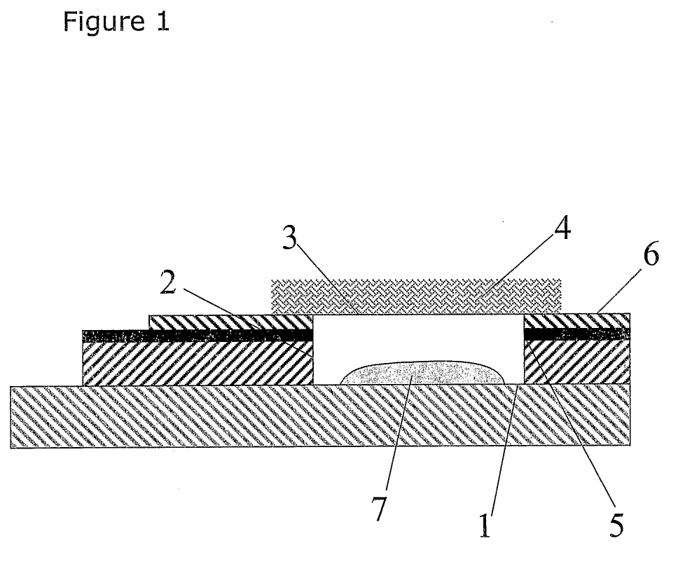

### DETAILED DESCRIPTION OF THE INVENTION

The present invention provides a method of selectively determining the HDL-cholesterol content of a sample, wherein the sample may contain other lipoproteins which bind to cholesterol, as well as HDL. Selectivity is achieved by reacting the sample with a specific surfactant which is highly selective for HDL over LDL, VLDL and CM. Thus, the surfactant makes available for measurement the cholesterol and cholesterol esters bound to HDL, whilst those bound to LDL, VLDL and CM remain bound to the lipoprotein structure and substantially do not react in the later measurement of the cholesterol content.

It is known that cholesterol and cholesterol esters are largely carried in the blood in lipoprotein particles. It is a matter of some debate as to the precise mechanism by which enzymes and surfactants enable such cholesterols to be made available to be oxidized by dehydrogenases. Therefore, throughout this specification, it is understood that terms such as ‘breaks down’ or ‘make available’ or ‘impart reactivity’ all relate to the process by which an analytic response is obtained from cholesterol(s) in any sample. However, we do not wish to be bound by any particular theory as to the mode of action. Surfactants which are said to ‘preferentially break down high density lipoproteins’ may therefore act, for example, by enhancing cholesterol availability preferentially in high density lipoproteins, or by a different mechanism.

The surfactants employed in the present invention are those which preferentially break down high density lipoproteins in a sample. This means that the surfactant reacts preferentially with HDL compared with LDL, VLDL and CM. The surfactants can alternatively be defined as those which selectively enable HDL cholesterol to react in a cholesterol assay, typically to react with a cholesterol ester hydrolysing reagent and cholesterol oxidase or dehydrogenase. In the context of the present invention, a surfactant which preferentially breaks down HDL, or a surfactant which selectively enables HDL cholesterol to react in a cholesterol assay, is typically a surfactant having a differentiation between HDL and LDL of at least 50%, preferably at least 60%, at least 70%, at least 80% or most preferably at least 90%.

The differentiation between HDL and LDL can be determined from data over a range of physiological samples by regression according to the equation (i):

$$Differentiation ( \% ) = \frac{{G}_{HDL} - {G}_{LDL}}{{G}_{HDL}} \times 100 \qquad ( i )$$

wherein G_{X} is the gradient of the measured response to X (e.g. the measured current vs the known concentration of X). The measured response may be any measured value which relates (or corresponds) to the lipoprotein concentration, for example which is proportional to the lipoprotein concentration.

In physiological samples both HDL and LDL cholesterols are present. In this case standard methods for multiple regressions may be used according to (i) to estimate the values of the respective gradients of the measured cholesterol concentration G_{HDL} and G_{LDL}.

The skilled person can therefore easily determine whether any given surfactant is one which preferentially breaks down HDL by using the chosen surfactant to measure the HDL cholesterol content of a sample of known HDL cholesterol content, and correspondingly measuring the LDL cholesterol content of a sample of known LDL cholesterol content using the same procedure. The differentiation value can be calculated from the results. The procedure for measuring the HDL or LDL cholesterol contents is typically that described in Example 1 below, using the chosen surfactant.

In the present invention, the concentration of HDL is typically measured electrochemically by determining the current generated at an electrode on electrochemical conversion of cholesterol to cholestenone. The measured current value is therefore typically used to determine the gradient.

In a preferred embodiment, the selective surfactants of the invention substantially do not break down LDL. Therefore, the differentiation between HDL and LDL is constant over time. However, some surfactants may still break down LDL, albeit very slowly. In this case, the differentiation between HDL and LDL may vary over time. The differentiation should be measured using a time lapse between addition of reagents to the sample and measurement of the cholesterol content which is the same as the time lapse to be used during HDL cholesterol testing. Such time lapse is typically in order of 3 minutes or less, preferably 120 seconds or less, 90 seconds or less or 60 seconds or less. In the context of the invention, a selective surfactant is typically a surfactant having a differentiation between HDL and LDL of at least 50%, preferably at least 60%, at least 70%, at least 80% or most preferably at least 90%, when measuring the cholesterol contents using the procedure described in Example 1 and a time lapse between addition of reagents to the sample and measurement of 62 seconds.

Variation of the differentiation can be seen from one physiological sample to another. This is readily observed as statistical variation or scatter about the calibration line of measured response against cholesterol concentration and can be expressed numerically as the R squared value (R²) or alternatively in terms of standard errors, for example the residual standard error of the multiple regression to HDL and LDL concentrations (Residual SE).

A particular advantage of the invention is the improvement provided in this variation, for example expressed as an increased R² value. Accordingly the method of the invention can be repeated one or more times for a single sample to generate a data set of a plurality of measurements relating to the HDL concentration of that sample, and the variation between the measurements in the data set is small. Typically, R² for any such data set is at least 0.6, preferably at least 0.7 or at least 0.8. The measurements in the data set may be measurements of HDL concentration, but more typically are measurements of a different parameter which is relates to, e.g. is proportional to, the HDL concentration. Typically, measurements of current generated in an electrochemical measurement are used.

This technique may be used, for example, in calibrating a device and the sample used in this case is one of known HDL concentration. R² in this case indicates the goodness of fit of the calibration model. Alternatively a data set may be generated in testing a sample of unknown HDL concentration (for example to provide an averaged final result), in which case the R² value indicates the reduced variability between the different measurements.

The surfactants for use in the invention include alkyl and cycloalkyl-alkyl hydroxyethyl glucamides of formula (I):

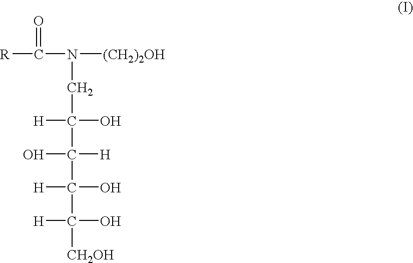

wherein R is an alkyl or cycloalkyl-alkyl group containing up to 12 carbon atoms. Such compounds include octanoyl-N-hydroxyethyl glucamide (R═CH₃(CH₂)₆, HEGA-8, available from Anatrace), decanoyl-N-hydroxyethyl glucamide (R═CH₃(CH₂)₈, HEGA-10, available from Anatrace), nonanoyl-N-hydroxyethyl glucamide (R═CH₃(CH₁₂)₇, HEGA-9), cyclohexylpropanoyl-N-hydroxyethyl glucamide (R═C₆H₁₁(CH₂)₂, C-HEGA-9, available from Anatrace) and cyclohexylbutanoyl-N-hydroxyethyl glucamide (R═C₆H₁₁(CH₂)₃, C-HEGA-10, available from Anatrace).

Further examples of surfactants of the invention include N-acyl-N-methyl glucamine derivatives of formula (II):

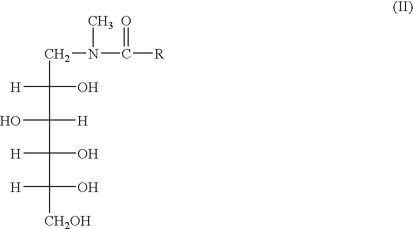

wherein R is an alkyl or cycloalkyl-alkyl group, typically an alkyl group, containing up to 12 carbon atoms. Examples of R groups are groups of formula —(CH₂)_{y}—CH₃ wherein y is from 5 to 11, e.g. 7, 8 or 9. Examples of such compounds include N-methyl-N-octanoyl-glucamine (MEGA-8), N-methyl-N-nonanoyl-glucamine (MEGA-9) and N-methyl-N-decanoyl-glucamine (MEGA-10).

Surfactants may be used singly or two or more different surfactants may be used in combination. The total amount of surfactant used is typically up to 200 mg per ml of sample to be tested, preferably up to 100 mg/ml, for example about 50 mg/ml.

If desired, the sample may additionally be reacted with a complexing agent which forms a complex with lipoproteins other than HDL. Examples of complexing agents include polyanions, combinations of polyanions with divalent metal salts, and antibodies capable of binding to apoB containing lipoproteins. The polyanions may be selected from phosphotungstic acid and salts thereof, dextran sulphuric acid and salts thereof, polyethylene glycol and heparin and salts thereof. Once in complexed form, the lipoproteins other than HDL are unavailable for reaction and therefore do not interfere with the cholesterol measurement. However, due to the use of specific surfactants in the present invention, complexing agents are not required. It is accordingly preferred that the sample is not reacted with a complexing agent.

Ionic salts may be used in combination with a selective surfactant. Examples of ionic salts are alkali metal (e.g. Na⁺, K⁺), alkaline earth (e.g.: Mg²⁺, Ca²⁺) or transition metal (e.g. Cr³⁺ salts. Chlorides are suitable salts. Use of an ionic salt can speed tip the kinetics of the reaction, so that maximum HDL differentiation is observed more quickly.

The measurement of the HDL-cholesterol content of the sample may be carried out by any suitable technique for measuring cholesterol. A preferred technique involves the reaction of the sample with a cholesterol ester hydrolysing reagent and cholesterol oxidase or cholesterol dehydrogenase. In one embodiment, cholesterol dehydrogenase is used, so the invention encompasses a method in which the sample is reacted with the surfactant and cholesterol dehydrogenase.

The cholesterol contained in HDL lipoproteins may be in the form of free cholesterol or cholesterol esters. A cholesterol ester hydrolysing reagent is therefore typically used to break down any cholesterol esters into free cholesterol. The free cholesterol is then reacted with the cholesterol oxidase or cholesterol dehydrogenase and the amount of cholesterol which has undergone such reaction is measured.

The cholesterol ester hydrolysing reagent may be any reagent capable of hydrolysing cholesterol esters to cholesterol. The reagent should be one which does not interfere with the reaction of cholesterol with cholesterol oxidase or cholesterol dehydrogenase and any subsequent steps in the assay. Preferred cholesterol ester hydrolysing reagents are enzymes, for example cholesterol esterase and lipases. A suitable lipase is, for example, a lipase from a pseudomonas or chromobacterium viscosum species. Commercially available enzymes, optionally containing additives such as stabilisers or preservatives may be used, e.g. those available from Toyobo or Amano. The cholesterol ester hydrolysing reagent may be used in an amount of from 0.1 to 20 mg per ml of sample, preferably from 0.5 to 15 mg per ml.

Any commercially available forms of cholesterol oxidase and cholesterol dehydrogenase may be employed. For instance, the cholesterol dehydrogenase is, for example, from the Nocardia species. The cholesterol oxidase or cholesterol dehydrogenase may be used in an amount of from 0.01 mg to 100 mg per ml of reagent mixture. In one embodiment, the cholesterol oxidase or dehydrogenase is used in an amount of from 0.1 to 25 mg per nil of sample, for example from 0.1 to 20 mg per ml of sample, preferably from 0.5 to 25 mg per ml, such as from 0.5 to 15 mg per ml.

Each of the enzymes may contain additives such as stabilisers or preservatives. Further, each of the enzymes may be chemically modified.

The surfactant may be added to the sample prior to addition of the other reagents or simultaneously with the addition of the other reagents. In a preferred embodiment, the cholesterol ester hydrolysing reagent, cholesterol oxidase or dehydrogenase and surfactant are present in a single reagent mixture which is combined with the sample in a single step. In a particularly preferred embodiment, the method involves a single step of contacting the sample with reagents, so that only a single reagent mixture need be provided.

Measurements in accordance with the present invention can be carried out on any suitable sample containing HDL-cholesterol. Measurements are typically carried out on whole blood or blood components, for example serum or plasma. Preferred samples for use in the method of the present invention are serum and plasma. Where measurements are to be carried out on whole blood, the method may include the additional step of filtering the blood to remove red blood cells.

In a preferred embodiment of the invention, an electrochemical technique is used to measure the HDL-cholesterol content. This means that the amount of cholesterol which has reacted with the cholesterol oxidase or cholesterol dehydrogenase is determined by measuring an electrochemical response occurring at an electrode. In this embodiment, the sample is typically reacted with the surfactant, a cholesterol ester hydrolysing reagent, cholesterol oxidase or cholesterol dehydrogenase, a coenzyme capable of interacting with cholesterol oxidase or cholesterol dehydrogenase, and a redox agent which is capable of being oxidised or reduced to form a product which can be electrochemically detected at an electrode. The mixture of sample and reagents is contacted with a working electrode of an electrochemical cell so that redox reactions occurring can be detected. A potential is applied across the cell and the resulting electrochemical response, typically the current, is measured.

In this preferred embodiment, the amount of HDL-cholesterol is measured in accordance with the following assay:

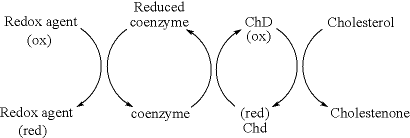

where ChD is cholesterol dehydrogenase. Cholesterol dehydrogenase could be replaced with cholesterol oxidase in this assay if desired. The amount of reduced redox agent produced by the assay is detected electrochemically. Additional reagents may also be included in this assay if appropriate.

Typically, the sample contacts all of the reagents in a single step. Therefore, a reagent mixture is provided which contains all of the required reagents and which can easily be contacted with the sample in order to carry out the assay. The reagent mixture of the invention typically comprises the surfactant in an amount of up to 50 mg, preferably up to 20 mg, for example about 5 mg per ml of sample, cholesterol ester hydrolysing reagent in an amount of from 0.1 to 20 mg, preferably from 0.5 to 10 mg per ml of sample and cholesterol dehydrogenase in an amount of from 0.1 to 30 mg, preferably from 0.5 to 25 mg per ml of sample.

Typically the coenzyme is NAD⁺ or an analogue thereof. An analogue of NAD⁺ is a compound having structural characteristics in common with NAD⁺ and which also acts as a coenzyme for cholesterol dehydrogenase. Examples of NAD⁺ analogues include APAD (Acetyl pyridine adenine dinucleotide); TNAD (Thio-NAD); AHD (acetyl pyridine hypoxanthine dinucleotide); NaAD (nicotinic acid adenine dinucleotide); NHD (nicotinamide hypoxanthine dinucleotide); and NGD (nicotinamide guanine dinucleotide). The coenzyme is typically present in the reagent mixture in an amount of from 1 to 20 mM, for example from 3 to 15 mM, preferably from 5 to 10 mM.

Typically, the redox agent should be one which can be reduced in accordance with the assay shown above. In this case, the redox agent should be one which is capable of accepting electrons from a coenzyme (or from a reductase as described below) and transferring the electrons to an electrode. The redox agent may be a molecule or an ionic complex. It may be a naturally occurring electron acceptor such as a protein or may be a synthetic molecule. The redox agent will typically have at least two oxidation states.

Preferably, the redox agent is an inorganic complex. The agent may comprise a metallic ion and will preferably have at least two valencies. In particular, the agent may comprise a transition metal ion and preferred transition metal ions include those of cobalt, copper, iron, chromium, manganese, nickel, osmium or ruthenium. The redox agent may be charged, for example it may be cationic or alternatively anionic. An example of a suitable cationic agent is a ruthenium complex such as Ru(NH₃)₆³⁺, an example of a suitable anionic agent is a ferricyanide complex such as Fe (CN)₆³⁻.

Examples of complexes which may be used include Cu(EDTA)²⁻, Fe(CN)₆³⁻, Fe(CN)₅(O₂CR)³⁻, Fe(CN)₄(oxalate)³⁻, Ru(NH₃)₆³⁺, Ru(acac)₂(Py-3-CO₂H)(Py-3-CO₂) and chelating amine ligand derivatives thereof (such as ethylenediamine), Ru(NH₃)₅(py)³⁺, ferrocenium and derivatives thereof with one or more of groups such as —NH₂, —NHR, —NHC(O)R, and —CO₂H substituted into one or both of the two cyclopentadienyl rings. Preferably the inorganic complex is Fe(CN)₆³⁻, Ru(NH₃)₆³⁺, or ferrocenium monocarboxylic acid (FMCA). Ru(NH₃)₆³⁺ and Ru(acac)₂(Py-3-CO₂H)(Py-3-CO₂) are preferred.

The redox agent is typically present in the reagent mixture in an amount of from 10 to 200 mM, for example from 20 to 150 mM, preferably from 30 to 100 mM, e.g. up to 80 mM.

In a preferred embodiment, the reagent mixture used in the electrochemical assay additionally comprises a reductase. The reductase typically transfers two electrons from the reduced NAD and transfers two electrons to the redox agent. The use of a reductase therefore provides swift electron transfer.

Examples of reductases which can be used include diaphorase and cytochrome P450 reductases, in particular, the putidaredoxin reductase of the cytochrome P450_{cam} enzyme system from Pseudomonas putida, the flavin (FAD/FMN) domain of the P450_{BM-}3 enzyme from Bacillus megaterium, spinach ferrodoxin reductase, rubredoxin reductase, adrenodoxin reductase, nitrate reductase, cytochrome b₅ reductase, corn nitrate reductase, terpredoxin reductase and yeast, rat, rabbit and human NADPH cytochrome P450 reductases. Preferred reductases for use in the present invention include diaphorase and putidaredoxin reductases.

The reductase may be a recombinant protein or a naturally occurring protein which has been purified or isolated. The reductase may have been mutated to improve its performance such as to optimise the speed at which it carries out the electron transfer or its substrate specificity.

The reductase is typically present in the reagent mixture in an amount of from 0.5 to 100 mg/ml, for example from 1 to 50 mg/ml, 1 to 30 mg/ml or from 2 to 20 mg/ml.

In a preferred embodiment of the invention, the general scheme of the electrochemical assay is as follows:

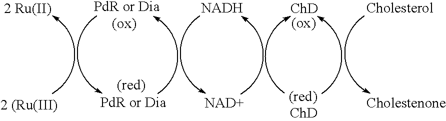

Where
PdR—is putidaredoxin reductase
Dia—is diaphorase
ChD—is cholesterol dehydrogenase.

The reagent mixture optionally contains one or more additional components, for example excipients and/or buffers and/or stabilisers. Buffers may also be included to provide the required pH for optimal enzyme activity. For example, a Tris buffer (pH9) may be used. Stabilisers may be added to enhance, for example, enzyme stability. Examples of suitable stabilisers are amino acids, e.g. glycine, and ectoine. Excipients may be included in the reagent mixture in order to stabilize the mixture and optionally, where the reagent mixture is dried onto the device of the invention, to provide porosity in the dried mixture. Examples of suitable excipients include sugars such as mannitol, inositol and lactose, and PEG. Glycine can also be used as an excipient. A particular advantage of the surfactants described herein is their combined action as surfactants and excipients. In one embodiment of the invention, therefore, the hydroxyethyl glucamide derivative and/or N-acyl-N-methyl glucamine derivative is used as a combined surfactant and excipient. In this embodiment, therefore a separate excipient is not used.

In a preferred embodiment, the reagent mixture for the electrochemical assay of the invention comprises a surfactant which preferentially breaks down high density lipoproteins; cholesterol esterase or a lipase; cholesterol dehydrogenase; NAD⁺ or an analogue thereof; a reductase; and a redox agent. In a more preferred embodiment, the reagent mixture comprises a surfactant which preferentially breaks down high density lipoproteins, cholesterol esterase or a lipase, cholesterol dehydrogenase, NAD⁺ or an analogue thereof, diaphorase or putidaredoxin reductase and Ru(NH₃)₆³⁺.

The reagent mixture of the invention is typically provided in solid form, for example in dried form, or as gel. Alternatively it may be in the form of a solution or suspension. Whilst the amounts of each of the components present in the reaction mixture are expressed above in terms of molarity or w/v, the skilled person would be able to adapt these amounts to suitable units for a dried mixture or gel, so that the relative amounts of each component present remains the same.

Where an electrochemical measurement is carried out on whole blood, the measurement obtained may depend on the hematocrit. The measurement should therefore ideally be adjusted to at least partially account for this factor. Alternatively, the red blood cells can be removed by filtering the sample prior to carrying out the assay.

The present invention also provides a kit for selectively determining the HDL cholesterol content of an HDL-containing sample. The kit includes the required reagents, e.g. the surfactant, cholesterol ester hydrolysing reagent and cholesterol oxidase or cholesterol dehydrogenase, as well as means for measuring the amount of cholesterol which reacts with the oxidase or dehydrogenase.

In a preferred embodiment, the kit comprises a device for the electrochemical determination of the HDL-cholesterol content. In this embodiment, the means for determining the amount of cholesterol which has reacted includes an electrochemical cell having a working electrode, a reference electrode or pseudo reference electrode and optionally a separate counter electrode; a power supply for supplying a potential across the cell; and a measuring instrument for measuring the resulting electrochemical response, typically the current across the cell.

The device for electrochemical determination typically includes a reagent mixture as described above. The reagents may be present in the kit individually or in the form of one or more reagent mixtures. A single regent mixture is preferred. The reagent mixture may be present in the device in either liquid or solid form, but is preferably in solid form.

Typically, the reagent mixture is inserted into or placed onto the device whilst suspended/dissolved in a suitable liquid (e.g. water or buffer) and then dried in position. This step of drying the material into/onto the device helps to keep the material in the desired position. Drying may be carried out, for example, by air-drying, vacuum drying, freeze drying or oven drying (heating), preferably by freeze drying. The reagent mixture is typically located in the vicinity of the electrodes, such that when the sample contacts the reagent mixture, contact with the electrodes also occurs.

The device may optionally comprise a membrane through which the sample to be tested passes prior to contact with the reagent mixture. The membrane may, for example, be used to filter out components such as red blood cells, erythrocytes and/or lymphocytes. Suitable filtration membranes, including blood filtration membranes, are known in the art. Examples of blood filtration membranes are Presence 200 and PALL BTS SP300 of Pall filtration, Whatman VF2, Whatman Cyclopore, Spectral NX and Spectral X. Fibreglass filters, for example Whatman VF2, can separate plasma from whole blood and are suitable for use where a whole blood specimen is supplied to the device and the sample to be tested is plasma.

Alternative or additional membranes may also be used, including those which have undergone a hydrophilic or hydrophobic treatment prior to use. Other surface characteristics of the membrane may also be altered if desired. For example, treatments to modify the membrane's contact angle in water may be used in order to facilitate flow of the desired sample through the membrane. The membrane may comprise one, two or more layers of material, each of which may be the same or different. For example, conventional double layer membranes comprising two layers of different membrane materials may be used.

Appropriate devices for use in the present invention include those described in WO 2003/056319 and WO 2006/000828.

A device according to one embodiment of the invention is depicted in FIG. 4. In this embodiment, the working electrode 5 is a microelectrode. For the purposes of this invention, a microelectrode is an electrode having at least one working dimension not exceeding 50 μm.

The cell is in the form of a receptacle or a container having a base 1 and a wall or walls 2. Typically, the receptacle will have a depth (i.e. from top to base) of from 25 to 1000 μm. In one embodiment, the depth of the receptacle is from 50 to 500 μm, for example from 100 to 250 μm. In an alternative embodiment, the depth of the receptacle is from 50 to 1000 μm, preferably from 200 to 800 μm, for example from 300 to 600 μm. The length and width (i.e. from wall to wall), or in the case of a cylindrical receptacle the diameter, of the receptacle is typically from 0.1 to 5 mm, for example 0.5 to 1.5 mm, such as 1 mm.

The open end of the receptacle 3 may be partially covered by an impermeable material or covered by a semi-permeable or permeable material, such as a semi-permeable or permeable membrane. Preferably, the open end of the receptacle is substantially covered with a semi-permeable or permeable membrane 4. The membrane 4 serves, inter alia, to prevent dust or other contaminants from entering the receptacle.

The working electrode 5 is situated in a wall of the receptacle. The working electrode is, for example, in the form of a continuous band around the wall(s) of the receptacle. The thickness of the working electrode is typically from 0.01 to 25 μm, preferably from 0.05 to 15 μm, for example 0.1 to 20 μm. Thicker working electrodes are also envisaged, for example electrodes having a thickness of from 0.1 to 50 μm, preferably from 5 to 20 μm. The thickness of the working electrode is its dimension in a vertical direction when the receptacle is placed on its base. The thickness of the working electrode is its effective working dimension, i.e. it is a dimension of the electrode which contacts the sample to be tested. The working electrode is preferably formed from carbon, palladium, gold or platinum, for example in the form of a conductive ink. The conductive ink may be a modified ink containing additional materials, for example platinum and/or graphite and/or platinised carbon. Two or more layers may be used to form the working electrode, the layers being formed of the same or different materials.

The cell also contains a pseudo reference electrode (not depicted) which may be present, for example, in the base of the receptacle, in a wall or walls of the receptacle or in an area of the device surrounding or close to the receptacle. The pseudo reference electrode is typically made from Ag/AgCl, although other materials may also be used. Suitable materials for use as the pseudo reference electrode will be known to the skilled person in the alt. In this embodiment, the cell is a two-electrode system in which the pseudo reference electrode acts as both counter and reference electrodes. Alternative embodiments in which the cell comprises a reference electrode and a separate counter electrode can also be envisaged.

The pseudo reference (or reference) electrode typically has a surface area which is of a similar size to or smaller than, or which is larger than, for example substantially larger than, that of the working electrode 5. Typically, the ratio of the surface area of the pseudo reference (or reference) electrode to that of the working electrode is at least 1:1, for example at least 2:1 or at least 3:1. A preferred ratio is at least 4:1. The pseudo reference (or reference) electrode may, for example, be a macroelectrode. Preferred pseudo reference (or reference) electrodes have a dimension of 0.01 mm or greater, for example 0.1 mm or greater. This may be, for example, a diameter of 0.1 mm or greater. Typical areas of the pseudo reference (or reference) electrode are from 0.001 mm² to 100 mm², preferably from 0.1 mm² to 60 mm², for example from 1 mm² to 50 mm². The minimum distance between the working electrode and the pseudo reference (or reference) electrode is, for example from 50 to 1000 μm. In order that the cell can operate, the electrodes must each be separated by an insulating material 6. The insulating material is typically a polymer, for example, an acrylate, polyurethane, PET, polyolefin, polyester or any other stable insulating material. Polycarbonate and other plastics and ceramics are also suitable insulating materials. The insulating layer may be formed by solvent evaporation from a polymer solution. Liquids which harden after application may also be used, for example varnishes. Alternatively, cross-linkable polymer solutions may be used which are, for example, cross-linked by exposure to heat or UV or by mixing together the active parts of a two-component cross-linkable system. Dielectric inks may also be used to form insulating layers where appropriate. In an alternative embodiment, an insulating layer is laminated, for example thermally laminated, to the device.

The electrodes of the electrochemical cell may be connected to any required measuring instruments by any suitable means. Typically, the electrodes will be connected to electrically conducting tracks which are, or can be, themselves connected to the required measuring instruments.

The required reagents are typically contained within the receptacle, as depicted at 7 in FIG. 4. Typically, the reagents, in the form of a single reagent mixture, are inserted into the receptacle in liquid form and subsequently dried to help immobilise the composition. The reagent mixture, for example, may be air-dried, vacuum dried, freeze dried or oven-dried (heated), most preferably it is freeze dried.

The device of the present invention is operated by providing a sample to the device and enabling the sample to contact the reagent mixture. It is clear that sufficient time has to be allowed for plasma or blood to dissolve and react with the reagents in the mixture. Where plasma is added to a sensor containing freeze dried reagents, a time of approximately 20 s elapses between the application of the sample to the sensor and application of the applied potential of the cell. Where whole blood is used, this delay time may be longer to allow for blood cell removal, for example up to 5 minutes (for example the blood may pass through a filtration membrane before contacting the reagents). In one embodiment of the invention, plasma is mixed with the reagents prior to contact with the electrochemical cell and added to the cell with immediate application of potential. The potential is typically applied and the measurement read within a period of 10 seconds, typically 1-4 seconds.

Typically, where Ru(II) is the product to be detected at the working electrode, the potential applied to the cell is from 0.1V to 0.3V. Preferred applied potential is 0.15V. (All voltages mentioned herein are quoted against a Ag/AgCl reference electrode with 0.1M chloride). In a preferred embodiment, the potential is stepped first to a positive applied potential of 0.15V for a period of about 1 second. A negative potential of −0.4 to −0.6V is then applied when it is desired to measure the reduction current. The use of the double potential step is described in WO 03/097860, incorporated herein by reference. Where a different redox agent is used, the applied potentials can be varied in accordance with the potentials at which the oxidation/reduction peak occurs.

The electrochemical test of the invention therefore enables a measurement of HDL cholesterol to be made in a very short period of time, typically within about 5 minutes, preferably within 3 minutes, 2 minutes or even 1 minute from application of a sample to the device. Results may in some circumstances be available in as little as 15 or 30 seconds from application of a sample to the device.

### EXAMPLES

### Example 1

### HEGA-8 with Ru(NH₃)₆Cl₃

The aim of the experiment was to investigate the response to HDL of sensors containing HEGA-8 surfactant and Ru(NH₃)₆Cl₃ mediator.

β-Lactose Buffer solution was prepared containing 0.1M Tris buffer pH9.0, 30 mM KOH, 10% β-Lactose. HEGA-8 solution was made up by addition of HEGA-8 to the lactose buffer solution to provide a final concentration of HEGA-8 of 5%.

Enzyme mixture was made by addition of enzymes to the HEGA-8 solution to provide the following final concentrations:
80 mM Ru(NH₃)₆Cl₃ (Alfa Aesar, 10511)
8.8 mM Thionicotinamide adenine dinucleotide (Oriental Yeast Co)
4.2 mg/ml Putidaredoxin Reductase (Biocatalyst)
3.3 mg/ml Lipase (Genzyme, from Chromobacterium viscosum)
22.2 mg/ml Cholesterol Dehydrogenase, Gelatin free (Amano, CHDH-6)

This solution was mixed using a Covaris acoustic mixer.

### Dispense and Freeze Drying

0.4 μL/well of each solution was dispensed onto the sensors using an electronic pipette. The dispensed sensor sheets were then freeze dried. The sensors were as described in WO2003/056319.

### Plasma Samples

Plasma samples were defrosted for 30 minutes before being centrifuged for 5 minutes at 2900 RCF. Delipidated serum (Scipac, S139) was also used as a sample. The samples were analysed using a Space clinical analyser for TC, TG, HDL and LDL concentrations.

### Testing Protocol

12-15 μl of a plasma sample was used per electrode. On the addition of plasma the chronoamperometry test was initiated. The oxidation current is measured at 0.15 V at 13 time points (0, 32, 64, 96, 128, 160, 192, 224, 256, 288, 320, 352 and 384 seconds), with a reduction current measured at −0.45 V at the final time point (416 seconds). Each sample was tested in duplicate.

### Analysis

These data were correlated to the HDL and LDL concentrations of the plasma samples from the space analyser. The gradients of response (measured current vs known concentration) to HDL and LDL at each time point were used to calculate the % differentiation obtained between measurement of LDL and HDL.

### Results

At 63 seconds, the gradients of response to HDL and L DL were 139.6 and 0.75 nA/mM respectively. The % differentiation to HDL was 99.5%.

High differentiation to HDL was obtained with sensors containing 5% HEGA-8 and 80 mM Ru(NH₃)₆Cl₃ mediator.

### Example 2

### C-HEGAs

The aim of the experiment was to investigate the response to HDL of sensors containing C-HEGA surfactants with different alkyl chain lengths.

β-Lactose Buffer solution was prepared containing 0.1M Tris buffer pH9.0, 30 mM KOH and 10% β-lactose. 30 mM RuAcac (Cis-[Ru(acac)₂(Py-3-CO₂H)(Py-3-CO₂)]) solution was made up by addition of RuAcac to the 10% lactose buffer. This solution was mixed using a Covaris acoustic mixer.

### C-HEGA Solutions

Double strength C-HEGA solutions were made by addition of C-HEGA to RuAcac solution to produce the following final concentrations:

### C-HEGA-8 (Anatrace, C408)

200 mM (0.0145 g in 207 μl RuAcac solution)
100 mM (50 μl of 200 mM stock+50 μl RuAcac solution)
50 mM (25 μl of 200 mM stock+75 μl RuAcac solution)

### C-HEGA-9 (Anatrace, C409)

200 mM (0.0148 g in 204 μl RuAcac solution)
100 mM (50 μl of 200 mM stock+50 μl RuAcac solution)
50 mM (25 μl of 200 mM stock+75 μl RuAcac solution)

### C-HEGA-10 (Anatrace, C410)

200 mM (0.0146 g in 193 μl RuAcac solution)
100 mM (50 μl of 200 mM stock+50 μl RuAcac solution)
50 mM (25 μl of 200 mM stock+75 μl RuAcac solution)

### C-HEGA-11 (Anatrace, C411)

200 mM (0.0155 g in 197 μl RuAcac solution)
100 mM (50 μl of 200 mM stock+50 μl RuAcac solution)
50 mM (25 μl of 200 mM stock+75 μl RuAcac solution)

### Enzyme Mixture

Enzyme mixture was made at double strength by addition of enzymes to RuAcac solution to produce the following final concentrations:

17.7 mM Thionicotinamide adenine dinucleotide (Oriental Yeast Co)
8.4 mg/ml Putidaredoxin Reductase (Biocatalyst)
6.7 mg/ml lipase (Genzyme, from Chromobacterium viscosum)
44.4 mpg/ml Cholesterol Dehydrogenase, Gelatin free (Amano, CHDH-6)
This solution was mixed using a Covaris acoustic mixer.

### Dispense and Freeze Drying

For each enzyme solution, equal volumes (approximately 50 uL) of double concentration enzyme solution and C-HEGA solutions were mixed 1:1 to give the final enzyme/surfactant mixes. In addition, a blank mix was prepared by mixing equal volumes (approximately 50 uL) each of double concentration enzyme solution and 30 mM RuAcac solution. 0.4 μL/well of each solution was dispensed onto the sensors using an electronic pipette. The dispensed sensor sheets were then freeze dried. The sensors used were as described in WO2003/056319.

Preparation of plasma samples, testing and analysis were as described in Example 1. The gradients of response to HDL and LDL at each time point were used to calculate the % differentiation obtained between measurement of LDL and HDL.

### Results

The gradients of response and % differentiation at 224 seconds are in the following table:

| | HDL | | |
|---|---|---|---|
| | gradient at | LDL | |
| | 224 sec/ | gradient at | % |
| sensor | nA/mM | 224 sec/nA/mM | differentiation |
| 100 mM C-HEGA-8 | 52.9 | 18.2 | 65.5 |
| 50 mM C-HEGA-8 | 50.9 | 17.4 | 65.9 |
| 25 mM C-HEGA-8 | 22.4 | 37.2 | −39.9 |
| 100 mM C-HEGA-9 | 46.6 | 27.1 | 41.8 |
| 50 mM C-HEGA-9 | 69.8 | 23.8 | 65.9 |
| 25 mM C-HEGA-9 | 43.0 | 24.3 | 43.6 |
| 100 mM C-HEGA-10 | 73.8 | 29.2 | 60.5 |
| 50 mM C-HEGA-10 | 53.8 | 31.0 | 42.4 |
| 25 mM C-HEGA-10 | 49.5 | 34.1 | 31.1 |
| 100 mM C-HEGA-11 | 92.5 | 36.6 | 60.4 |
| 50 mM C-HEGA-11 | 79.0 | 43.4 | 45.1 |
| 25 mM C-HEGA-11 | 37.3 | 37.8 | −1.3 |
| blank | 30.0 | 30.8 | −2.8 |

Compared to the sensor response with no added surfactant, the gradient of response and % differentiation to HDL are significantly increased by the use of these C-HEGA surfactants.

### Example 3

### HEGAs

The aim of the experiment was to investigate the response to HDL of sensors containing HEGA surfactants with different alkyl chain lengths.

Ru Acac solution was made up as described in Example 2.

### HEGA Solutions

Double strength HEGA solutions were made by addition of HEGA to RuAcac solution to produce the following final concentrations:

### HEGA-8 (Anatrace, H108)

200 mM (0.0149 g in 212 μl RuAcac solution)
1.00 mM (50 μl of 200 mM stock+50 μl RuAcac solution)
50 mM (25 μl of 200 mM stock+75 μl RuAcac solution)

### HEGA-9 (Anatrace, H109)

200 mM (0.0143 g in 196 μl RuAcac solution)
100 mM (50 μl of 200 mM stock+50 μl RuAcac solution)
50 mM (25 μl of 200 mM stock+75 μl RuAcac solution)

### Enzyme Mixture

Enzyme mixture was made at double strength by addition of enzymes to RuAcac solution to produce the following final concentrations:

17.7 mM Thionicotinamide adenine dinucleotide (Oriental Yeast Co)
8.4 mg/ml Putidaredoxin Reductase (Biocatalyst)
6.7 mg/ml Lipase (Genzyme, from Chromobacterium viscosum)
44.4 mg/ml Cholesterol Dehydrogenase, Gelatin free (Amano, CHDH-6)

This solution was mixed using a Covaris acoustic mixer, using Covaris S-series SonoLab-S1 software-Programme HDL 4° C.

Dispense, freeze drying, preparation of plasma samples, testing and analysis were carried out as described in Example 2.

### Results

The gradients of response and % differentiation at 96 seconds are in the following table:

| | HDL gradient at | LDL gradient at | % |
|---|---|---|---|
| sensor | 96 sec/nA/mM | 96 sec/nA/mM | differentiation |
| 100 mM HEGA-8 | 50.6 | 7.2 | 85.8 |
| 50 mM HEGA-8 | 58.2 | 8.8 | 84.9 |
| 25 mM HEGA-8 | 27.8 | 19.3 | 41.4 |
| 100 mM HEGA-9 | 103.9 | 13.3 | 87.2 |
| 50 mM HEGA-9 | 92.8 | 10.5 | 88.7 |
| 25 mM HEGA-9 | 71.5 | 6.1 | 91.5 |
| blank | 25.6 | 6.9 | 73.0 |

Compared to the sensor response with no added surfactant, the gradient of response and % differentiation to HDL are significantly increased by the use of these HEGA surfactants.

Further, the analysis by multiple regression for correlation between the response at 118 seconds and HDL and LDL concentrations are given in the following table.

| | | HDL: | HDL: | HDL: | HDL: | HDL: | HDL: |
|---|---|---|---|---|---|---|---|
| | | 100 mM | 50 mM | 25 mM | 100 mM | 50 mM | 25 mM |
| 96 sec | HDL: Blank | Hega 9 | Hega 9 | Hega 9 | Hega 8 | Hega 8 | Hega 8 |
| HDL Slope | 32.44 | 104.40 | 96.83 | 74.62 | 51.45 | 60.44 | 45.06 |
| (nA/mM) | | | | | | | |
| LDL Slope | 8.86 | 4.89 | 4.62 | 3.35 | 3.58 | 7.43 | 14.50 |
| (nA/mM) | | | | | | | |
| HDL Slope | 18.31 | 27.81 | 20.36 | 11.91 | 20.07 | 23.11 | 10.65 |
| SE | | | | | | | |
| LDL Slope | 3.55 | 5.40 | 3.95 | 2.31 | 4.14 | 4.76 | 2.20 |
| SE | | | | | | | |
| R Squared | 0.67 | 0.75 | 0.83 | 0.89 | 0.54 | 0.61 | 0.91 |
| Residual SE | 12.18 | 18.49 | 13.54 | 7.92 | 13.81 | 15.89 | 7.32 |
| % Diff | 72.70 | 95.32 | 95.23 | 95.51 | 93.04 | 87.70 | 67.83 |

### Example 4

### Reproducibility with Glucamide

The aim of the experiment was to demonstrate improved correlation of measured response to plasma HDL concentration, when using a glucamide surfactant compared to TC specific surfactants or no surfactants.

10% β-Lactose Buffer was as described in Example 1.

### Surfactant Solutions

Double strength surfactant solutions were made up as follows:

CHAPS (Sigma-Aldrich Co. Ltd, C5070) 10% 0.1996 g in 1.996 mls 10% lactose buffer
DeoxybigCHAPS (Soltec Ventures, S115) 10% 0.1763 g in 1.763 mls CHAPS solution Hega 9 (Anatrace, H109) 200 mM 0.1257 g in 1.719 mls 10% lactose buffer

RuAcac was then added to buffer solution and to each surfactant solution to provide final concentrations of 30 mM RuAcac in each:

0.0248 g RuAcac in 1.512 mls 10% lactose buffer=Blank (no surfactant)
0.0266 g RuAcac in 1.622 mls 10% CHAPS/10% DeoxybigCHAPS solution
0.0267 g RuAcac in 1.628 mls 200 mM Hega 9 solution
Ru Acac mediator=Cis-[Ru(acac)₂(Py-3-CO₂H)(Py-3-CO₂)]

These solution were mixed using a Covaris acoustic mixer.

### Enzyme Mixture

Enzyme mixture was made at double strength by addition of enzymes to blank RuAcac solution to produce the following final concentrations:

17.7 mM Thionicotinamide adenine dinucleotide (Oriental Yeast Co)
8.4 mg/ml Putidaredoxin Reductase (Biocatalyst)
20.2 mg/ml Lipase (Genzyme, from Chromobacterium viscosum)
44.4 mg/ml Cholesterol Dehydrogenase, Gelatin free (Amano, CHDH-6)

This solution was mixed using a Covaris acoustic mixer.

### Production Dispense and Freeze Drying

For each enzyme solution, equal volumes (approximately 1.4 mls) of double concentration enzyme solution and surfactant solutions were mixed 1:1 to give the final enzyme/surfactant mixes. In addition, a blank mix was prepared by mixing equal volumes (approximately 1.4 mls) each of double concentration enzyme solution and blank 30 mM RuAcac solution. The sensors used were as described in WO2003/056319. 0.35 μL/well of each solution was dispensed onto the sensors and freeze dried.

Plasma samples were prepared as described in Example 2.

### Testing Protocol

12-15 μl of a plasma samples was used per electrode. On the addition of plasma the chronoamperometry test was initiated. The oxidation current was measured at 0.15 V at 8 time points (0, 59, 118, 177, 236, 295, 354 and 413 seconds), with a reduction current measured at −0.45 V at the final time point (472 seconds). Each sample was tested 8 times.

### Analysis

These data were correlated to the HDL concentrations of the plasma samples from the space analyser. Calibration plots for response to HDL at each time point were constructed. Data from all sensors was combined.

### Results

The correlation coefficients for the calibration plots for each sensor type at 59 sec are given in the following table:

| Sensor type | Correlation coefficient |
|---|---|
| No added surfactant | 0.43 |
| 5% CHAPS, 5% deoxy bigCHAP | 0.29 |
| 100 mM HEGA-9 | 0.71 |

Higher correlation is obtained between measured sensor response and HDL concentration for sensors containing HEGA-9, compared to sensors with no added surfactant or with CHAPS/deoxy bigCHAP.

Further, the analysis by multiple regression for correlation between the response at 118 seconds and HDL and LDL concentrations are given in the following table.

| | | HDL | LDL | HDL | LDL St | Residual | |
|---|---|---|---|---|---|---|---|
| 118 Sec | R sq | Grad | Grad | St Err | Err | St Err | % Diff |
| No surfactant: | 0.871 | 37.935 | 14.240 | 5.678 | 2.752 | 8.576 | 62.46 |
| Multianalyte for | | | | | | | |
| reproducibility | | | | | | | |
| 100 mM HEGA- | 0.866 | 64.942 | 4.097 | 7.604 | 3.685 | 11.485 | 93.69 |
| 9: Multianalyte for | | | | | | | |
| reproducibility | | | | | | | |
| 5% CHAPS, | 0.819 | 27.403 | 16.765 | 6.388 | 3.096 | 9.649 | 38.82 |
| 5% deoxy bigCHAP: | | | | | | | |
| Multianalyte for | | | | | | | |
| reproducibility | | | | | | | |

### Example 5

### HEGA-9 & Ionic Salts

The aim of the experiment was to investigate the response to HDL of sensors containing 100 mM HEGA-9 and various ionic salts.

10% β-Lactose Buffer was prepared as described in Example 1.

mM RuAcac made up by addition of RuAcac to 10% lactose buffer. This solution was mixed using a Covaris acoustic mixer.

HEGA-9 solution was made up by addition of 0.2612 g HEGA-9 (Anatrace, H109) to 3.724 mL RuAcac solution.

### Ionic Salt Solutions

Ionic salt solutions were prepared at double concentration by addition of salts to RuAcac solution to produce the following final concentrations:

### LiCl (Aldrich, 21, 323-3)

1.5 M LiCl solution: 0.0113 grams were dissolved in 177 μL of HEGA-9 solution.

1 M LiCl solution: 40 μL of 1.5 M LiCl solution was mixed with 20 μL of HEGA-9 solution.

### NaCl (Sigma, S-7653)

1 M NaCl solution: 0.0066 grams were dissolved in 113 μL of HEGA-9 solution.

### MgCl₂ (Sigma, C-4901)

500 mM MgCl₂ solution: 0.0103 grams were dissolved in 101 μL of HEGA-9 solution.

250 mM MgCl₂ solution: 30 uL of 500 mM MgCl₂ solution was mixed with 30 uL of HEGA-9 solution.

### CaCl₂ (Sigma, M2670)

500 mM CaCl₂ solution: 0.0058 grams were dissolved in 105 μL of HEGA-9 solution.

250 mM CaCl₂ solution: 30 uL of 500 mM CaCl₂ solution was mixed with 30 uL of HEGA-9 solution.

### Cr(NH₃)₆Cl₃ (Manchester Organics)

120 mM Cr(NH₃)₆Cl₃ solution: 0.0075 grams were dissolved in 120 μL of HEGA-9 solution.

### Co(NH₃)₆Cl₃ (Alfa Aesar, A15470)

120 mM Co(NH₃)₆Cl₃ solution: 0.0082 grams were dissolved in 127 μL of HEGA-9 solution.

60 mM CoNH₃)₆Cl₃: 30 μL of 120 mM Co(NH₃)₆Cl₃ solution was mixed with 30 μL of HEGA-9 solution.

### Enzyme Mixture

Enzyme mixture was made at double strength by addition of enzymes of HEGA-9 solution to produce the following final concentrations:

17.7 mM Thionicotinamide adenine dinucleotide (Oriental Yeast Co)
8.4 mg/ml Putidaredoxin Reductase (Biocatalyst)
6.7 mg/ml Lipase (Genzyme)
44.4 mg/ml Cholesterol Dehydrogenase, Gelatin free (Amano, CHDH-6)

This solution was mixed using a Covaris acoustic mixer.

### Dispense and Freeze Drying

For each enzyme solution, equal volumes (approximately 40 uL) of double concentration enzyme solution and ionic salt solutions were mixed 1:1 to give the final enzyme/ionic salt mixes. In addition, a blank mix was prepared by mixing equal volumes (approximately 40 uL) each of double concentration enzyme solution and 30 mM HEGA-9 solution. 0.4 μL/well of each solution was dispensed onto sensors as described in WO2003/056319 using an electronic pipette, and freeze dried.

Plasma samples were prepared as described in Example 2.

### Testing Protocol

12-15 μl of a plasma samples was used per electrode. On the addition of plasma the chronoamperometry test was initiated. The oxidation current is measured at 0.15 V at 8 time points (0, 59, 118, 177, 236, 295, 354 & 413 seconds), with a reduction current measured at −0.45 V at the final time point (472 seconds). Each sample was tested in duplicate.

These data were correlated to the HDL and LDL concentrations of the plasma samples from the space analyser. The gradients of response to HDL and LDL at each time point were used to calculate the % differentiation obtained between measurement of LDL and HDL.

### Results

The time at which the gradient of response to HDL reached maximum value was seen to vary between the enzyme mixes. The times are given in the table below:

| | time at which HDL |
|---|---|
| | gradient is maximum/ |
| sensor | sec |
| 100 mM HEGA-9 | 236 |
| 100 mM HEGA-9 &750 mM | 177 |
| LiCl | |
| 100 mM HEGA-9 &500 mM | 177 |
| LiCl | |
| 100 mM HEGA-9 &500 mM | 177 |
| NaCl | |
| 100 mM HEGA-9 &125 mM | 177 |
| CaCl2 | |
| 100 mM HEGA-9 &125 mM | 118 |
| MgCl2 | |
| 100 mM HEGA-9 &250 mM | 177 |
| MgCl2 | |
| 100 mM HEGA-9 &60 mM | 177 |
| Cr(NH3)6Cl3 | |
| 100 mM HEGA-9 &30 mM | 118 |
| Co(NH3)6Cl3 | |

For sensors prepared with 100 mM HEGA-9, addition of ionic salt resulted in the maximum gradient of response to HDL being reached more quickly if ionic salt was present in the enzyme mix. In other words, the kinetics of response were increased by the presence of an ionic salt.

### Example 6

### HEGA-9 Titration and Different Lipase or ChE

The aim of the experiment was to investigate the dependence of the response to HDL of sensors prepared with a wide range of concentrations of HEGA-9, and also to investigate the effect of different lipase or cholesterol esterase.

Two separate enzyme mixes were made on the same day, one for the HEGA-9 titration and one for the use of Toyobo lipase or Genzyme cholesterol esterase.

### Enzyme Mix for HEGA-9 Titration:

30 mM Ruacac buffer was made up as described in Example 2

### HEGA-9 Solutions

Double strength HEGA-9 solutions were made by addition of HEGA-9 to RuAcac solution to produce the following final concentrations:

### HEGA-9 (Anatrace, H109)

600 mM (0.0550 g in 251 μl RuAcac solution)
400 mM (60 μl of 600 mM stock+30 μl RuAcac solution)
200 mM (30 μl of 600 mM stock+60 μl RuAcac solution)
100 mM (15 μl of 600 mM stock+75 μl RuAcac solution).
500 mM (10 μl of 600 mM stock+110 μl RuAcac solution)
20 mM (3 μl of 600 mM stock+87 μl RuAcac solution)

### Enzyme Mixture

Enzyme mixture was made at double strength by addition of enzymes to RuAcac solution to produce the following final concentrations:

17.7 mM Thionicotinamide adenine dinucleotide (Oriental Yeast Co)
8.4 mg/ml Putidaredoxin Reductase (Biocatalyst)
6.7 mg/ml Lipase (Genzyme)
44.4 mg/ml Cholesterol Dehydrogenase, Gelatin free (Amano, CHDH-6)

This solution was mixed using a Covaris acoustic mixer.

For each enzyme solution, equal volumes (approximately 50 uL) of double concentration enzyme solution and HEGA-9 solutions were mixed 1:1 to give the final enzyme/surfactant mixes. In addition, a blank mix was prepared by mixing equal volumes (approximately 50 uL) each of double concentration enzyme solution and 30 mM RuAcac solution.

### Enzyme Mix for Toyobo Lipase or Genzyme Esterase:

Lactose buffer solution was made up as described in Example 1. 100 mM HEGA-9 solution was made up by addition of 0.0372 g HEGA-9 (Anatrace, H109) to 1.018 mls of lactose buffer solution. RuAcac solution was made up by addition of 0.0158 g RuAcac to 963 uL of 100 mM HEGA-9 solution.

### Enzyme Mixtures

Two separate final enzyme mixes were prepared with either lipase from Pseudomonas sp. (Toyobo) or cholesterol esterase from Pseudomonas sp. (Genzyme).

Enzyme mixtures were made at single strength by addition of enzymes to RuAcac solution to produce the following final concentrations:

8.8 mM Thionicotinamide adenine dinucleotide (Oriental Yeast Co)
4.2 mg/ml Putidaredoxin Reductase (Biocatalyst)
3.3 mg/ml Lipase (Toyobo) or 3.3 mg/mL Cholesterol esterase (Genzyme)
22.2 mg/ml Cholesterol Dehydrogenase, Gelatin free (Amano, CHDH-6)

The solutions were mixed using a Covaris acoustic mixer.

Dispense, freeze drying, plasma sample preparation, testing and analysis were carried out as described in Example 2.

### Results

The gradients of response and % differentiation at 118 seconds are in the following table:

| | HDL | LDL | |
|---|---|---|---|
| | gradient at | gradient at | % |
| sensor | 118 sec/nA/mM | 118 sec/nA/mM | differentiation |
| 300 mM HEGA-9 | 29.7 | 12.1 | 59.3 |
| 200 mM HEGA-9 | 36.8 | 9.1 | 75.3 |
| 100 mM HEGA-9 | 44.1 | 4.4 | 90.1 |
| 50 mM HEGA-9 | 37.3 | 1.5 | 96.0 |
| 25 mM HEGA-9 | 43.7 | 17.0 | 61.0 |
| 10 mM HEGA-9 | 31.6 | 16.5 | 47.7 |
| blank | 27.2 | 13.9 | 48.8 |
| 100 mM | 47.8 | 18.8 | 60.7 |
| HEGA-9 & | | | |
| Toyobo lipase | | | |
| 100 m | 47.1 | −2.4 | 105.0 |
| HEGA-9 & | | | |
| Genzyme esterase | | | |

Compared to the sensor response with no added surfactant, the gradient of response and % differentiation to HDL are significantly increased by the use of HEGA-9 surfactant. The optimal amount of HEGA-9 is 50-200 mM, for highest differentiation to HDL.

Compared to the sensor response with no added surfactant, sensors with 100 mM HEGA-9 and either Genzyme lipase, Toyobo lipase or Genzyme esterase give increased gradient of response and differentiation to HDL.

### Example 7

### MEGAs

The aim of the experiment was to investigate the response to HDL of sensors containing MEGA surfactants with different alkyl chain lengths.

Example 2 was repeated using MEGA-7 and MEGA-8 solutions.

Ru Acac solution was made up as described in Example 2

### MEGA Solutions

Double strength MEGA solutions were made by addition of MEGA to RuAcac solution to produce the following final concentrations:

MEGA-7 (Heptanoyl-N-methyl glucamide) (Sigma H1639)
200 mM (0.0178 g in 290 μw RuAcac solution)
100 mM (50 μl of 200 mM stock+50 μl RuAcac solution)
50 mM (25 μl of 200 mM stock+75 μl RuAcac solution)

### MEGA-8 (Soltec Ventures S116)

200 mM (0.0188 g in 292 μl RuAcac solution)
100 mM (50 μl of 200 mM stock+50 μl RuAcac solution)
50 mM (25 μl of 200 mM stock+75 μl RuAcac solution)

### Enzyme Mixture

Enzyme mixture was made at double strength by addition of enzymes to RuAcac solution to produce the following final concentrations:

17.7 mM Thionicotinamide adenine dinucleotide (Oriental Yeast Co)
8.4 mg/ml Putidaredoxin Reductase (Biocatalyst)
6.7 mg/ml Lipase (Genzyme)
44.4 mg/ml Cholesterol Dehydrogenase, Gelatin free (Amano, CHDH-6)

This solution was mixed using a Covaris acoustic mixer.

### Results

The gradients of response and % differentiation at 224 seconds are in the following table:

| | HDL | | |
|---|---|---|---|
| | gradient at | LDL | |
| | 224 sec/ | gradient at | % |
| sensor | nA/mM | 224 sec/nA/mM | differentiation |
| 100 mM MEGA-7 | 86.0 | 16.0 | 81.0 |
| 50 mM MEGA-7 | 60.0 | 19.0 | 69.0 |
| 25 mM MEGA-7 | 49.0 | 19.0 | 61.0 |
| 100 mM MEGA-8 | 72.0 | 41.0 | 43.0 |
| 50 mM MEGA-8 | 54.0 | 21.0 | 61.0 |
| 25 mM MEGA-8 | 59.0 | 21.0 | 65.0 |
| blank | 33.0 | 20.0 | 39.0 |

Compared to the sensor response with no added surfactant, the gradient of response and % differentiation to HDL are significantly increased by the use of these MEGA surfactants.

### Example 8

### Blank & HEGA-9 Variability

The aim of the experiment was to investigate the variability in response to plasma HDL observed with different batches of sensors prepared with either no added surfactant or 100 mM HEGA-9.

Sensors were prepared on several occasions over a period of several months.

The final formulations are given below for the blank and HDL chemistries.

### Final Enzyme Mix Containing No Added Surfactant

### 0.1 M Tris (pH 9.0)

### 30 mM KOH

10% w/v lactose

### 30 mM RuAcac

8.8 mM Thionicotinamide adenine dinucleotide (Oriental Yeast Co)
4.2 mg/ml Putidaredoxin Reductase (Biocatalyst)
3.3 mg/ml Lipase (Genzyme, Chromobacterium viscosum)
22.2 mg/ml Cholesterol Dehydrogenase, Gelatin free (Amano, CHDH-6)

### Final Enzyme Mix Containing 100 Mm HEGA-9

### 0.1 M Tris (pH 9.0)

### 30 mM KOH

10% w/v lactose

### 30 mM RuAcac

### 100 mM HEGA-9 (Anatrace, H109)

8.8 mM Thionicotinamide adenine dinucleotide (Oriental Yeast Co)
4.2 mg/ml Putidaredoxin Reductase (Biocatalyst)
3.3 mg/ml Lipase (Genzyme, Chromobacterium viscosum)
22.2 mg/ml Cholesterol Dehydrogenase, Gelatin free (Amano, CHDH-6)

Dispense and Freeze Drying were as described in Example 2.

### Testing Protocol

12-15 μl of a plasma samples was used per sensor. On the addition of plasma the chronoamperometry test was initiated. The oxidation current is measured at 0.15 V at 13 time points (0, 32, 64, 96, 128, 160, 192, 224, 256, 288, 320, 352 and 384 seconds), with a reduction current measured at −0.45 V at the final time point (416 seconds). The transient time was 4 seconds. Each sample was tested in duplicate.

Some calibrations were performed with 8 second transient times. The oxidation current is measured at 0.15 V at either 6 or 8 time points (0, 59, 118, 177, 236, 295, (354), (413) seconds), with a reduction current measured at −0.45 V at the final time point (354 or 472 seconds). Each sample was tested in duplicate.

These data were analysed, along with the HDL and LDL concentrations of the plasma samples from the Space clinical analyser. Multiple linear regression was performed to obtain the HDL and LDL gradients of response and the intercept at each time point. The % differentiation was obtained at each time point using the gradients of response.

### Results

For each calibration, the r² value and the % differentiation are tabulated below for the time point at which the correlation coefficient (r² value) was highest for the current vs. [HDL] plot.

| | meas | | |
|---|---|---|---|
| | time/ | | |
| chemistry | sec | r\hat{ }2 | % diff |
| 100 mM | 256 | 0.62 | 94.5 |
| HEGA-9 | | | |
| 100 mM | 96 | 0.68 | 88.4 |
| HEGA-9 | | | |
| 100 mM | 236 | 0.68 | 86 |
| HEGA-9 | | | |
| 100 mM | 118 | 0.72 | 93.7 |
| HEGA-9 | | | |
| 100 mM | 177 | 0.62 | 73.5 |
| HEGA-9 | | | |
| 100 mM | 118 | 0.78 | 100.5 |
| HEGA-9 | | | |
| 100 mM | 295 | 0.75 | 92.1 |
| HEGA-9 | | | |
| Standard deviation of | | | 8.56 |
| differentiation | | | |
| Average r\hat{ }2 | | 0.69 | |
| n = 7 | | | |
| blank | 384 | 0.17 | 73.6 |
| blank | 384 | 0.48 | 80.1 |
| blank | 384 | 0.41 | 79.6 |
| blank | 384 | 0.08 | 64.6 |
| blank | 354 | 0.47 | 80.9 |
| blank | 413 | 0.69 | 80.2 |
| blank | 384 | 0.62 | 87.8 |
| blank | 384 | 0.19 | 40.2 |
| blank | 384 | 0.20 | 51.8 |
| blank | 384 | 0.67 | 89.4 |
| blank | 384 | 0.20 | 59.5 |
| blank | 384 | 0.43 | 80.3 |
| blank | 384 | 0.68 | 91.7 |
| Standard deviation of | | | 15.46 |
| differentiation | | | |
| Average r\hat{ }2 | | 0.41 | |
| n = 13 | | | |

The average values and the standard deviation of the r² and the % differentiation are shown for blank and HEGA-9 sensors.

The average r² value and % differentiation were significantly higher for sensors with 100 mM HEGA-9, compared to sensors with no added surfactant. In addition the standard deviation of the r² value and % differentiation values were lower for the sensors with 100 mM HEGA-9 compared to the sensors with no added surfactant.

Use of 100 mM HEGA-9 significantly increases the correlation of the response of sensors to plasma HDL concentration, compared to sensors with no added surfactant. In addition the batch to batch reproducibility of the sensor response to plasma HDL is significantly increased by the use of HEGA-9.

1. A method for the determination of the amount of cholesterol in high density lipoproteins in a high density lipoprotein containing sample, said method comprising reacting the sample with (a) a surfactant which preferentially breaks down high density lipoproteins, said surfactant being selected from hydroxyethyl glucamide derivatives and N-acyl-N-methyl glucamine derivatives, and measuring the amount of cholesterol in the high density lipoproteins.

2. A method according to claim 1, wherein the surfactant is for improving the reproducibility of the determination of the amount of cholesterol in high density lipoproteins.

3. A method according to claim 1, wherein the surfactant (a) has a differentiation for HDL over LDL given by the equation (i) below of at least 50%:
$$Differentiation ( \% ) = \frac{{G}_{HDL} - {G}_{LDL}}{{G}_{HDL}} \times 100 \\ wherein {G}_{HDL} is \frac{measured ( HDLcholesterol )}{known [ HDLcholesterol ]} , \\ and \\ {G}_{LDL} is \frac{measured ( LDLcholesterol )}{known [ LDLcholesterol ]} . \qquad ( i )$$
wherein measured (HDL cholesterol) or measured (LDL cholesterol) is a measured value which relates to the concentration of HDL or LDL respectively.

4. A method according to claim 1, wherein the surfactant (a) is selected from HEGA-8, HEGA-9, HEGA-10, C-HEGA-9, C-HEGA-10, MEGA-8, MEGA-9, MEGA-10 and MEGA-12.

5. A method according to claim 1, wherein the method comprises repeating the measurement one or more times for a given sample to provide a data set of a plurality of measurements relating to the HDL concentration of the sample, and wherein R² for the data set is at least 0.6.

6. A method according to claim 1, wherein the amount of cholesterol in the high density lipoproteins is measured by reacting the sample with (b) a cholesterol ester hydrolysing reagent and (c) cholesterol oxidase or cholesterol dehydrogenase and determining the amount of cholesterol which has reacted with the cholesterol oxidase or cholesterol dehydrogenase.

7. A method according to claim 1, wherein the amount of cholesterol in the high density lipoproteins is measured by an electrochemical technique.

8. A method according to claim 7, wherein the method comprises reacting the sample with
(a) a surfactant which preferentially breaks down high density lipoproteins;
(b) a cholesterol ester hydrolysing reagent;
(c) cholesterol oxidase or cholesterol dehydrogenase;
(d) a coenzyme; and
(e) a redox agent capable of being oxidised or reduced to form a product;
and electrochemically detecting the amount of product formed.

9. A method according to claim 8, wherein the sample is additionally reacted with (f) a reductase.

10. A method according to claim 1, wherein the sample is reacted simultaneously with the surfactant (a), the cholesterol ester hydrolysing reagent (b) and the cholesterol oxidase or cholesterol dehydrogenase (c).

11. A method according to claim 1, wherein the high density lipoprotein containing sample is whole blood and wherein the method additionally comprises the step of filtering the sample to remove red blood cells.

12. A method according to claim 1, wherein the measurement of the amount of cholesterol in the high density lipoproteins is completed within a period of no more than 3 minutes from reaction of the sample with the surfactant.

13. A reagent mixture for use in a method for the determination of the amount of cholesterol in high density lipoproteins in a high density lipoprotein containing sample, the reagent mixture comprising
(a) a surfactant which preferentially breaks down high density lipoproteins, said surfactant being selected from hydroxyethyl glucamide derivatives and N-acyl-N-methyl glucamine derivatives;
(b) a cholesterol ester hydrolysing reagent; and
(c) cholesterol oxidase or cholesterol dehydrogenase.

14. A reagent mixture according to claim 13, which additionally comprises (d)
a coenzyme, (e) a redox agent capable of being oxidised or reduced to form a product; and optionally (f) a reductase.

15. A kit for the determination of the amount of cholesterol in high density lipoproteins in a high density lipoprotein containing sample, the kit comprising (a) a surfactant which preferentially breaks down high density lipoproteins, said surfactant being selected from hydroxyethyl glucamide derivatives and N-acyl-N-methyl glucamine derivatives, (b) a cholesterol ester hydrolysing reagent, and (c) cholesterol oxidase or cholesterol dehydrogenase, and optionally one or more of (d) a coenzyme, (e) a redox agent capable of being oxidised or reduced to form a product, and (f) a reductase, and means for measuring the amount of cholesterol which reacts with the cholesterol oxidase or cholesterol dehydrogenase.

16. A kit according to claim 15 wherein the means for measuring the amount of cholesterol which reacts with the cholesterol oxidase or cholesterol dehydrogenase comprises
an electrochemical cell having a working electrode, a reference or pseudo reference electrode and optionally a separate counter electrode;
a power supply for applying a potential across the cell; and
a measuring instrument for measuring the resulting electrochemical response.

17. A kit according to claim 15, wherein the reagents (a), (b) and (c) and optionally one or more of (d), (e) and (f) are present in the form of a single reagent mixture.

18. A kit according to claim 17, wherein the reagent mixture is in dried form.

19. A method of operating a kit for the determination of the amount of cholesterol in high density lipoproteins in a high density lipoprotein containing sample, the kit comprising:
(a) a surfactant which preferentially breaks down high density lipoproteins, said surfactant being selected from hydroxyethyl glucamide derivatives and N-acyl-N-methyl glucamine derivatives, (b) a cholesterol ester hydrolysing reagent, and (c) cholesterol oxidase or cholesterol dehydrogenase, and optionally one or more of (d) a coenzyme, (e) a redox agent capable of being oxidised or reduced to form a product and (f) a reductase;
and means for measuring the amount of cholesterol which reacts with the cholesterol oxidase or cholesterol dehydrogenase comprising
an electrochemical cell having a working electrode, a reference or pseudo reference electrode and optionally a separate counter electrode;
a power supply for applying a potential across the cell; and
a measuring instrument for measuring the resulting electrochemical response; said method comprising
(i) contacting (1) the reagents (a), (b) and (c), and optionally one or more of (d), (e) and (f), and (2) a high density lipoprotein containing sample, with each other and with the electrodes;
(ii) applying a potential across the electrochemical cell; and
(iii) electrochemically detecting the amount of product formed by measuring the resulting electrochemical response.

20. A method according to claim 19, wherein step (iii) is completed within a period of up to 3 minutes from the time at which the sample is contacted with the reagents (a), (b) and (c).

21. A method of improving the reproducibility of a determination of the amount of cholesterol in high density lipoproteins, comprising reacting a high density lipoprotein containing sample with a surfactant selected from hydroxyethyl glucamide derivatives and N-acyl-N-methyl glucamine derivatives.
